(19) United States
(12) Patent Application Publication
(10) Pub. No.: US 2005/0261656 A1
(43) Pub. Date: Nov. 24, 2005

(54) Injury prevention apparatus

(76) Inventors: Mario C. Garcia, West St. Paul, MN (US); Walter H. Diers JR., Chaska, MN (US)

Correspondence Address: HAUGEN LAW FIRM, SUITE 1130 - TCF TOWER, 121 SOUTH EIGHTH STREET, MINNEAPOLIS, MN, 55402, US

(21) Appl. No.: 10/851,278

(22) Filed: May 21, 2004

(51) Int. Cl. A61F 13/15

(52) U.S. Cl. 604/385.12; 604/385.01

(57) ABSTRACT

A wearable article that is specifically configured for protecting certain user body parts from trauma as a result of external forces focused thereupon incorporates one or more force dispersal elements disposed at one or more predetermined protection locations of the wearable article for cushioning external forces focused at one or more of such particular user body regions correspondingly aligned with the one or more protection locations of the wearable article when it is properly worn by the user. In certain embodiments, the wearable article is an adult diaper having one or more force dispersal elements selectively disposed at particular locations thereof. In other embodiments, the wearable article incorporates one or more force dispersal elements that are disposed at a rear portion of the article specifically for protecting the user's coccyx region when the garment is properly worn.

### FIELD OF THE INVENTION

The present invention relates to protective elements for protecting certain body parts of a user from injury or irritation as a result of external forces imparted thereupon in, for example, falls from a standing position or continuous pressure from prolonged bed rest. The present invention relates more particularly to (a) reusable wearable garments having such protective elements selectively disposed thereat such that the design of the garment provides for the placement of the protective elements; (b) the design of protective elements to disperse or absorb external forces; (c) methods of attachment or placement of such protective elements; and (d) disposable wearable garments having protective elements that allow the selective incorporation thereof.

### BACKGROUND OF THE INVENTION

An ongoing issue in the field of geriatric health is the risk of injury to elderly individuals as a result of falls or other relatively minor impacts. In certain groups, such injuries can include bone fractures, which can lead to additional more serious health issues, particularly for certain increased risk individuals. Among some of these greater at risk groups are: persons, particularly females, over age fifty, persons with chronic medical conditions, persons with impaired acuity, persons with osteoporosis, and persons with bodies having low muscle/fat content. Common types of bone fractures sustained in a fall are focused at the hip (greater trochanter).

A further issue that finds relatively common occurrence in the geriatric population is the formation of decubitus ulcers, which are more commonly known as pressure sores or bed sores. Such ulcers are caused by prolonged pressure on the skin, particularly at locations of the body with prominent bones or cartilage. Any person who must sit or lie in a relatively immobile state for prolonged periods of time are susceptible to bed sores. The weight of the body in contact with a surface such as a bed or wheelchair seat places continuous pressure on certain areas of the body resulting in decreased blood flow to the skin in such areas. The reduced blood flow results in a breakdown of the skin and the formation of a decubitus ulcer. One of the most common areas in which a decubitus ulcer may occur is the coccyx region.

A variety of devices have been constructed to reduce the occurrence of hip injuries due to falls or related events. These devices include hip pads used by athletes while playing soccer, hockey, baseball, and also by skiers. Devices have also been constructed to prevent or reduce damage from such an injury among the elderly. Examples of such devices may be found in U.S. Pat. Nos. 5,717,997 and 6,195,809. Typically, such injury protection is provided via garments that incorporate in some manner the use of pads or padded regions placed at a portion of the garment that is aligned with the greater trochanter of the users. Thus, the pads so incorporated act to absorb, disperse, and deflect energy created in a fall and directed to the user's greater trochanter, thereby reducing the likelihood of serious injury to the user.

The garments proposed to date incorporating such protective devices, however, typically utilize a pocket that is integrally formed therewith, which pocket is sized and configured to enclose a specific pad therein. In addition, protective garments currently available are limited in their ability to modify the relative location of such protective devices in order to maximize their usefulness with users of various size. Conventional protective garments may therefore not be best suited for users of a size and shape different than model users identified and targeted in the manufacture of such garments. Additionally, currently available protective garments typically focus solely on fall protection at the hip area of the user, and fail to address other sources of injury or regions of injury, such as decubitus ulcers in the coccyx region.

An additional significant issue that is present primarily in the geriatric population is incontinence, with many members of this population using adult diapers as a result of incontinence problems. Such adult diapers are typically changed multiple times each day, and in cases where both diapers and conventional impact protective garments are utilized together, the time and effort required to repeatedly remove and replace such garments is a significant work load on users or caregivers. Since incontinence management is typically perceived as a need that is more important than impact protection, the use of protective devices is often times ignored, thereby placing those in need of such protection at a greater risk of injury. Incontinence also exacerbates the formation of decubitus ulcers, in that the acid present in urine and fecal matter tend to cause skin to weaken and breakdown.

A further issue with respect to current protective garments and their associated protective devices is the relative expense of manufacture and maintenance thereof. Typically, protective garments and protective devices incorporated therewith are reusable and thus washable. Such a design requires a relatively durable garment that is relatively expensive to manufacture. Incorporating disposable force dispersal elements into protective garments may result in a more affordable system.

It is therefore a principal object of the present invention to provide a modular system for providing force dispersion to a user at one or more predetermined injury-prone locations.

It is another object of the present invention to provide a protective garment incorporating one or more protective elements selectively disposed at predetermined locations thereof for protection of specific injury-prone regions of the user's body.

It is another object of the present invention to provide an adult diaper having one or more force dispersal elements selectively incorporated therewith for protection of specific injury-prone regions of the user's body.

It is a yet further object of the present invention to provide a protective garment that incorporates one or more protective elements specifically positioned at the garment to absorb and disperse forces focused at the coccyx region of the user.

It is a further object of the present invention to provide distinct and disposable protective elements for providing force dispersal at one or more predetermined injury-prone locations of the user.

It is another object of the present invention to provide attachment mechanisms that may be separate and distinct from the garment.

It is a still further object of the present invention to provide one or more reusable protective elements that may operate in conjunction with additional disposable protective elements for providing force dispersal at one or more predetermined injury-prone locations of the user.

### SUMMARY OF THE INVENTION

By means of the present invention, an injury prevention apparatus is provided for inhibiting and/or preventing injury to the user as a result of external forces being directed at specific injury-prone regions of the user's body. In particular, the present invention is directed at inhibiting and/or preventing injuries at the greater trochanter and coccyx region of respective users by incorporating force dispersal elements conveniently positioned at portions of the prevention apparatus which operably align with the injury-prone regions of the user when the prevention apparatus is properly worn. Moreover, the prevention apparatus may preferably be constructed as an adult diaper to provide both injury protection and incontinence care characteristics.

In a particular embodiment of the present invention, an adult diaper is provided for use in operably absorbing excretory materials from the user, with the adult diaper further being configured for providing force dispersal to particular body regions of the user. The adult diaper includes a main structure having an inner surface and an outer surface, and front and rear portions extending from a midsection thereof. The main structure is configured to be worn about the user's hips and between the user's legs via suspension elements operably coupled to the adult diaper. The adult diaper further includes force dispersal elements selectively disposed only at one or more predetermined protection locations of the main structure for cushioning external forces focused at one or more of such particular user body regions correspondingly aligned with the one or more protection locations of the main structure when the diaper is properly worn by the user.

Another embodiment of the present invention provides a wearable garment that is specifically configured for protecting certain user body parts from trauma as a result of external forces focused thereupon. The wearable garment includes force dispersal elements selectively disposed at a rear portion thereof that is adjacent to the user's coccyx region when the garment is properly worn. The force dispersal elements are embodied in one or more discrete packages distinctly positioned at the garment and are selected from one of foam pads, rubber pads, selectively inflatable air bladders, closed-cell air pocket sheets, gel pads, shields, and combinations thereof.

### BRIEF DESCRIPTION OF THE DRAWINGS

FIG. 1 is a side-elevational view of an injury prevention apparatus of the present invention being properly worn by a user;

FIG. 2 is a top plan view of an injury prevention apparatus of the present invention;

FIG. 3 is a top plan view of an injury prevention apparatus of the present invention.

FIG. 4 is a side-elevational view of an injury prevention apparatus of the present invention being properly worn by a user;

FIG. 5 is a isolation and side-elevational view of a pocket means of an injury prevention apparatus of the present invention;

FIG. 6 is a top plan view of an injury prevention apparatus of the present invention;

FIG. 7 is a top plan view of an injury prevention apparatus of the present invention;

FIG. 8 is a top plan view of an injury prevention apparatus of the present invention; and

FIG. 9 is a top plan view of an injury prevention apparatus of the present invention.

### DETAILED DESCRIPTION OF THE PREFERRED EMBODIMENTS

The objects and advantages enumerated above together with other objects, features, and advances represented by the present invention will now be presented in terms of detailed embodiments described with reference to the attached drawing figures which are intended to be representative of various possible configurations of the invention. Other embodiments and aspects of the invention are recognized as being within the grasp of those having ordinary skill in the art.

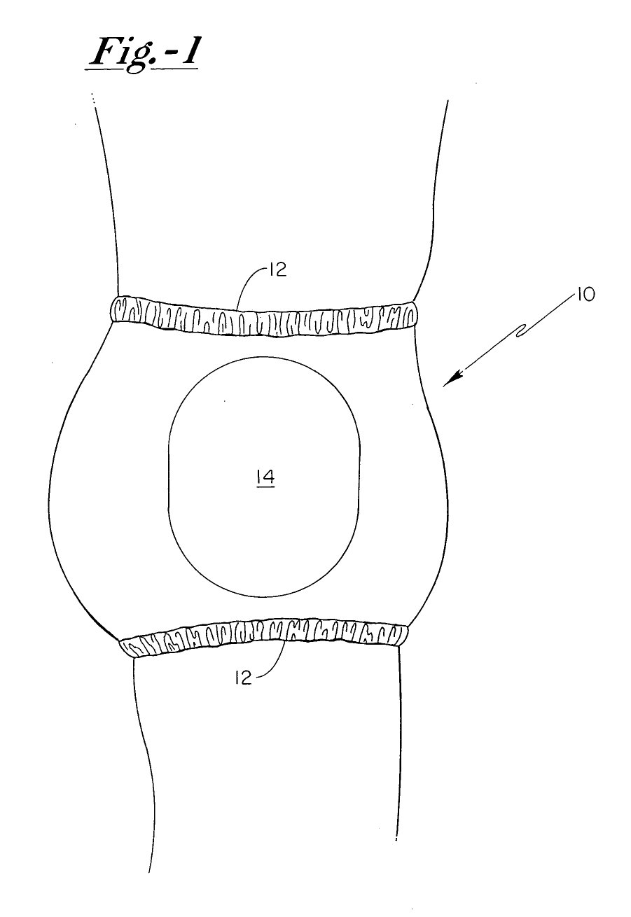

With reference to the enclosed drawing figures, and first to FIG. 1, a protective garment 10 is illustrated as being properly worn by the user, such that garment 10 is operably positioned about the user's hips and between the user's legs. To assist in retaining a desired positioning on the user's body, garment 10 preferably utilizes suspension means 12, which may be in the form of an integrated elastic band or the like. Garment 10, however, may instead be suspended and retained on the user via tape, mechanical fasteners, hook and loop fabric patches or the like. Other embodiments of garment 10 may incorporate both elastic band means and other fastener elements in combination with one another in order to securely position garment 10 upon the user. All of the above-described mechanisms for securing garment 10 in position on the user are well known in the art, and are referred to herein as suspension means.

In preferred embodiments of the present invention, garment 10 is an adult diaper that is capable of absorbing human excretory materials such as urine, blood, and fecal matter. The embodiment illustrated in FIG. 1 is an adult diaper that is held in place about the user's hips by suspension means 12, which may be one or both of an elastic band or tape means integrally formed with or selectively attached to garment 10. It is further contemplated by present invention to incorporate other types of suspension means 12 in absorbent garment 10.

Absorbent garment 10 preferably further includes force dispersal elements formed as force dispersal elements 14 disposed at one or more specific locations of garment 10 to provide protection to an adjacent body region of the user. A force dispersal element 14 is shown in FIG. 1 as being positioned at garment 10 so as to be operably aligned with the user's greater trochanter for impact protection thereof. As further described hereinbelow, force dispersal elements 14 may be disposed at locations of garment 10 other than in operable alignment with the greater trochanter of the user. An example of such an alternative or additional placement of force dispersal elements 14 in garment 10 is at a location thereof in operable alignment with the coccyx region of the user.

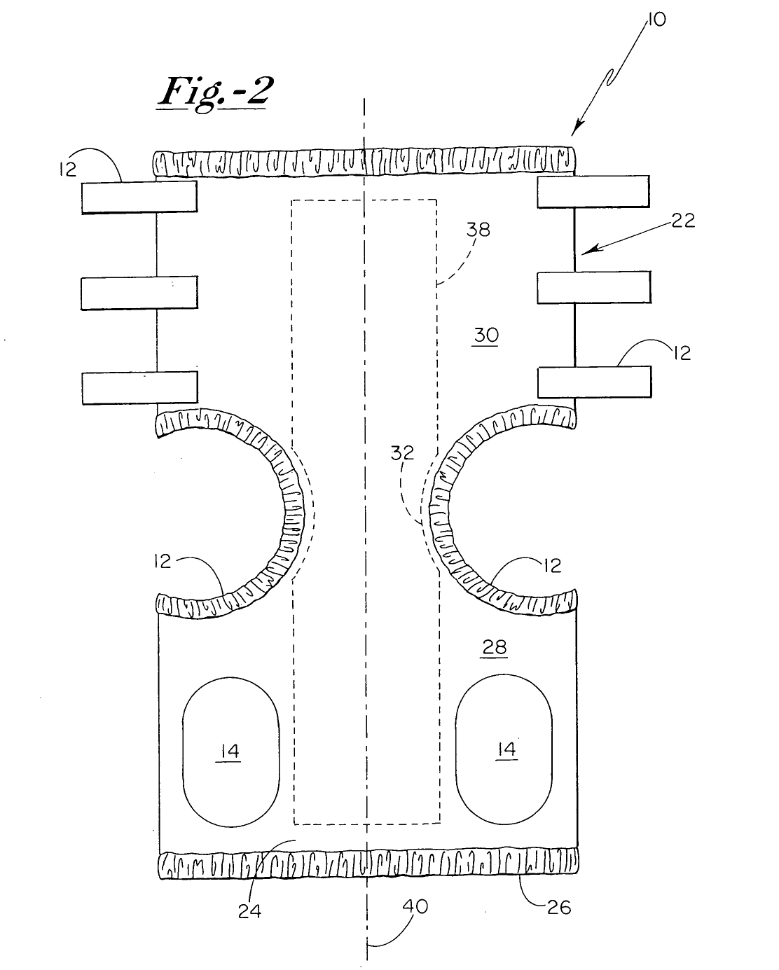

FIG. 2 illustrates an embodiment of absorbent garment 10 in an open orientation prior to being secured about the hips and between the legs of the user. Garment 10 preferably includes a main structure 22 having an outer surface 24 and an inner surface 26, and front and rear portions 28, 30 extending from a mid-section 32 thereof. In the embodiment illustrated in FIG. 2, suspension means 12 is in the form of tape, a portion of which is adhesively secured to either or both of outer or inner surfaces 24, 26 of main structure 22. Such tape suspension means and methods of attachment thereof are well known in the art, and are commonly utilized in adult diapers.

Main structure 22 is preferably constructed as a conventional adult diaper, with one or more layers encasing or supporting absorbent material in an absorbent region 38 thereof, which absorbent region 38 is typically centered over a bisecting longitudinal axis 40 of main structure 22 so as to be best positioned for quickly receiving excremental materials from the user. In such conventional adult diaper construction, an inner surface layer 26 is fabricated from a relatively soft liquid-pervious material to thereby allow fluidous excretions to pass to absorbent material disposed between inner surface layer 26 and outer surface layer 24. Moreover, outer surface layer 24 is preferably fabricated from a liquid-impervious material to thereby act as a barrier layer to prevent undesired leakage of excretory materials from absorbent garment 10.

Absorbent garment 10 further includes one or more distinct force dispersal elements 14 disposed at either or both of outer or inner surface layers 24, 26 of main structure 22. Such force dispersal elements 14 provide force dispersion means for absorbing external forces created in a fall or simply through the weight of the user upon a surface such as a chair or bed, and disperses such energy over a relatively large surface area to thereby minimize localized forces focused at injury-prone regions of the user, such as the greater trochanter and the coccyx region. Accordingly, force dispersal elements 14 are preferably disposed only at one or more predetermined locations of main structure 22 that operably align with particular user body regions that are susceptible to injury when absorbent garment 10 is properly worn by the user.

In the embodiments illustrated in FIGS. 1 and 2, one or more force dispersal elements 14 are preferably positioned at front and/or rear portions 28, 30 of main structure 22 so as to align with the greater trochanter of the user when absorbent garment 10 is operably folded upon itself about the hips and between the legs of the user. In such an orientation, front portion 28 may be coupled to rear portion 30 via a suspension means 12. A wide variety of other locations for force dispersal elements 14 on main structure 22 are contemplated by the present invention, including at a location of main structure 22 that is in operable alignment with the user's coccyx region when garment 10 is properly worn.

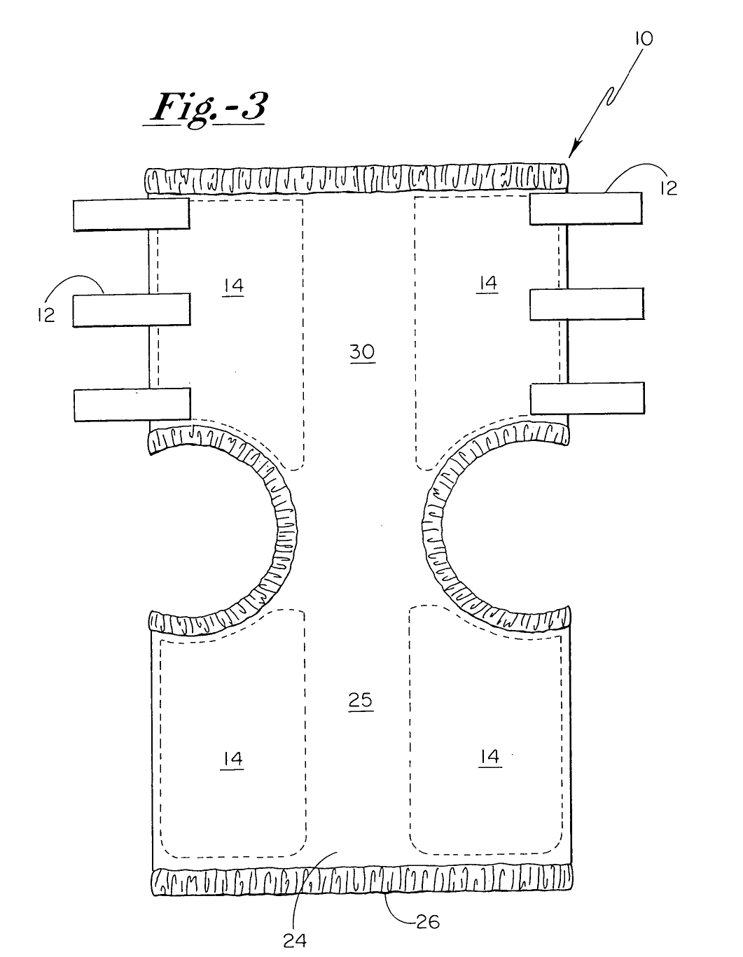

In the embodiment illustrated in FIG. 3, force dispersal elements 14 may be disposed at both front and rear portions 28, 30 of garment 10 such that when garment 10 is properly worn by the user, the force dispersal elements 14 disposed at front portion 28 overlap or underlap the force dispersal elements 14 at rear portion 30, thereby providing two layers of force dispersant material aligned over the specific injury-prone region of the user such as the greater trochanter.

Force dispersal elements 14 may be provided in a variety of shapes, sizes, and materials. Preferably, however, each of the force dispersal elements 14 are configured to provide adequate energy absorption and force dispersion upon the imposition of focused external forces, in order to minimize the risk of injury to the corresponding body regions of the user. As such, force dispersal elements 14 are preferably each at least 5 in.² in size and more preferably are each between about 10 in.² and about 60 in.² in size. Depending upon the materials utilized for respective force dispersal elements 14, the respective thickness thereof is typically between about 0.125 inches and about 2.0 inches, though other thicknesses may be employed as desired.

An example material for use in force dispersal elements 14 is a closed-cell air pocket sheet such as that commonly utilized in packaging applications, with each of the respective force dispersal elements 14 incorporating one or more air pocket sheets preferably having a plurality of distinct air pockets formed therein. Such closed-cell air pocket sheets may preferably be utilized as disposable force dispersal elements 14 that may be selectively and/or removably secured to garment 10 for temporary use thereof.

Closed-cell air pocket sheets are widely commercially available in desired thicknesses and roll lengths. Exemplary closed-cell air pocket sheets are available from Sealed Air Corporation of Saddlebrook, N.J. under the trade name “Air Cap®” high performance air cellular cushioning in 1/16″ to ½″ thickness or “Bubble Wrap®” general purpose air cellular cushioning in 1/16″ to ½″ thickness. Distinct force dispersal elements 14 are obtained by die-cutting the material into desired dimensions.

Other materials and combinations for use in force dispersal elements 14 are further contemplated by the present invention. Such materials and combinations include, for example, rubber pads, selectively inflatable air bladders, resilient polymeric foam, shields, gel pads, and combinations thereof. As stated above, some embodiments of the present invention may incorporate selectively inflatable air bladders either alone or in combination with other energy absorbing materials in the construction of force dispersal elements 14. In such a manner, the degree of firmness and/or thickness of the respective force dispersal elements 14 may be selectively adjusted by the user through selective inflation of the air bladders.

In other embodiments, one or more of force dispersal elements 14 may be a resilient polymeric foam material known in the art for absorbing energy from forces placed thereupon. In some embodiments, AFT #202, a closed-cell, cross-linked polyethylene available from American Flexible Products of Chaska, Minn., is used. Other closed-cell foam may be used as well. The AFT #202 foam has the following properties:

| Property | Value |
|---|---|
| Density (lbs/ft³) | |
| ASTM | 3575½ inch |
| Tensile Strength (psi) | 50 |
| Elongation (%) | 180 |
| Tear strength (lbs per inch) | 10.2 |
| Compression deflection (psi) | 7.8 |
| Compression Set (%) | 12 |
| K-Factor | 0.247 |
| Working temp (° F.) | −70 to 212 |
| Water absorption (psi) | 0.01 |

A further aspect and embodiment of force dispersal elements 14 is in the use of shields either used alone or in combination with other materials in the construction thereof. Such shields are preferably relatively rigid in order to effectively disperse external forces about an entire surface area of the respective force dispersal elements 14. Through such a dispersion of external forces, the degree of energy imparted upon the corresponding injury-prone body portion of the user is substantially reduced.

A particular example of a relatively rigid shield useful in force dispersal elements 14 of the present invention is one manufactured of a relatively rigid polymer or co-polymer such as ABS, polyurethane, and polypropylene. A wide variety of other materials, such as metals, may be incorporated in place of, or in combination with those materials identified above in the shields of the present invention. In embodiments where such shields are utilized in combination with other force dispersal elements in the construction of respective force dispersal elements 14, the shields and other material elements are preferably secured together through adhesives, hook and loop type fasteners, snap fasteners, respective mating flanges, and the like. The securement between respective components of such force dispersal elements 14 inhibits and otherwise prevents relative motion therebetween. In addition, such shields preferably conform to a generally convex curvature to most comfortably surround the injury-prone region of the user such as the greater trochanter.

Force dispersal elements 14 may be configured to be removably securable to respective portions of main structure 22. To enable such a removable securement characteristic, force dispersal elements 14 preferably include removable attachment means (not shown) such as one or more adhesive strips or patches, hook and loop type fasteners, and the like. The adhesive material utilized in conjunction with the removably securable force dispersal elements 14 may be, for example, a pressure sensitive adhesive disposed on a polymeric backing material, which may be in the form of tape strips. Such removable attachment means may be disposed at any desired location of respective force dispersal elements 14 to allow the removable securement of such force dispersal elements 14 in any desired orientation. In a particular embodiment of the invention, such removable attachment means is disposed on at least one major surface of the respective force dispersal elements 14 for removably securing such force dispersal elements 14 to a respective surface of main structure 22. In some embodiments, removable attachment means are provided on each opposed major surfaces of respective force dispersal elements 14 in order to removably secure such force dispersal elements 14 to both main structure 22 and an additional surface, such as a further force dispersal elements 14. A particular application of adhering two force dispersal elements to one another is in applying a shield element to a respective outer surface of a further material to obtain a multi-material force dispersal element 14, and to thereby enhance the injury protection and force dispersal characteristics provided by such force dispersal element 14.

Though certain embodiments of the present invention may incorporate garments 10 and/or force dispersal elements 14 that are reusable and therefore washable, it is particularly contemplated in the present invention to incorporate force dispersal elements 14 and garments 10 that are disposable and specifically manufactured for a single use. The disposable nature of the respective components of the present invention add to the overall ease of use thereof. Such ease of use is intended to encourage a more widespread acceptance and utilization of the system of the present invention over reusable embodiments. Accordingly, force dispersal elements 14 are preferably relatively inexpensive so as to be disposable by the end user after a single use.

In some embodiments of the present invention, one or more removable peel away layers may be provided in superimposed relationship over the attachment means on respective force dispersal elements 14 to thereby prevent undesired or premature adhesion of such force dispersal elements 14. In such a manner, such removable peel away layers are kept intact upon respective force dispersal elements 14 until the time at which the user desires to selectively secure such force dispersal elements 14 to main structure 22. At such time, the removable peel away layers are selectively removed by the user to expose the attachment means disposed thereunder, such that the respective force dispersal element 14 may be selectively secured to the intended target. Due to the surface characteristics of such removable peel away layers, the attachment means preferentially adhere to the respective force dispersal elements 14 to thereby enable selective detachment of such removable peel away layers therefrom.

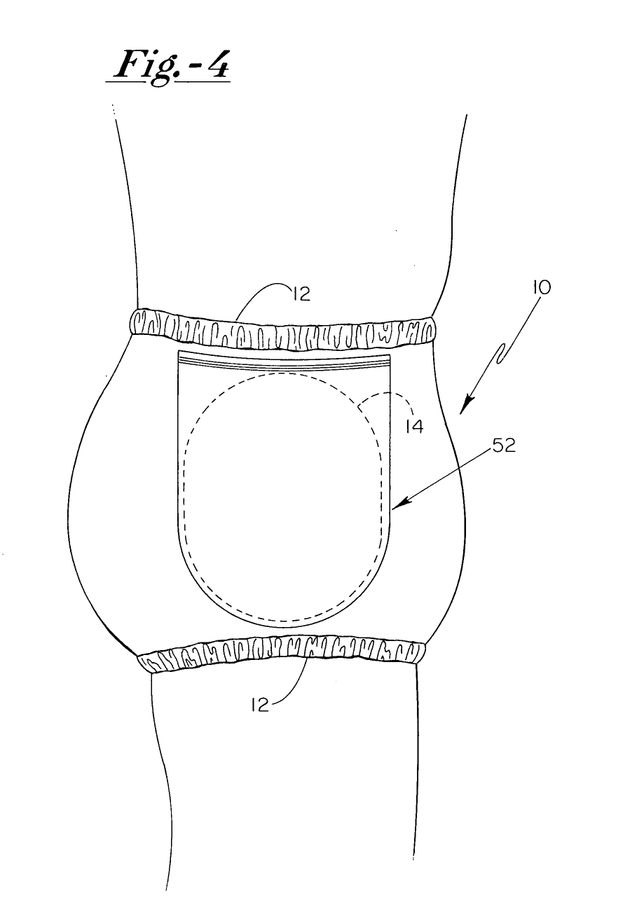

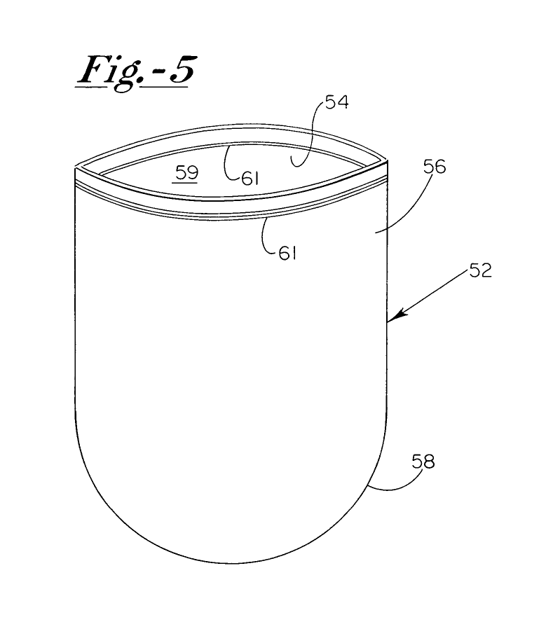

As illustrated in FIGS. 4-5, a pocket device 52 may be provided for operably receiving one or more force dispersal elements 14 therein. In preferred embodiments of the present invention, pocket device 52 is a distinct body having a first side member 54 and a second side member 56 that are engaged with one another at least partially about a perimeter 58 of pocket device 52. Therefore, pocket device 52 defines a cavity that is at least partially enclosed by first and second side members 54, 56.

A variety of configurational embodiments for pocket device 52 are contemplated by the present invention. For example, pocket device 52 may define a body that is closed on all but one side thereof, thereby providing an opening to the cavity defined by first and second side members 54, 56 for operably inserting and/or removing one or more force dispersal elements 14 into or from pocket device 52. In some embodiments, open end 59 of pocket device 52 may be selectively closed via a closure means 61. Such a closure means 61 may take the form of, for example, hook and loop type fastening elements, tongue and groove mating structures, folds in one or more of first and second side members 54, 56, and the like. Such closure means may be provided in pocket device 52 of the present invention to securely store one or more force dispersal elements 14 therein.

In still further embodiments of the present invention, pocket device 52 may be completely enclosed so as to define an enclosed cavity therewithin. In such embodiments, the one or more force dispersal elements 14 are permanently contained within pocket device 52. Thus, the combination of pocket device 52 and the one or more force dispersal elements 14 disposed therewithin may be configured to be disposable as a unit or reusable and washable as a unit.

In preferred embodiments of the present invention, pocket device 52 is fabricated from a liquid-impervious material such as a polymeric material. Examples of polymeric materials useful in the construction of pocket device 52 include low density polyethylene, high density polyethylene, and polypropylene, though other liquid-impervious materials may be incorporated in addition to, or in place of the above-identified polymeric materials. The liquid-impervious characteristic of pocket device 52 provides protection to the one or more force dispersal elements 14 disposed therewithin from exposure to liquids such as bodily excretions from the user. In such a manner, pocket device 52 may be exposed to liquids or other materials and be subsequently washed and reused or disposed of without detrimental impact to the corresponding force dispersal elements 14 where such force dispersal elements 14 are removably insertable into such pocket device 52. The above-described characteristic is particularly useful where the force dispersal elements 14 are fabricated from liquid-absorbent materials that could be destroyed or their functionality compromised if exposed to materials such as excretory substances from the user.

In addition, one or more pocket devices 52 may be selectively secured to main structure 22 of garment 10, particularly at locations corresponding to injury-prone regions of the user such as the greater trochanter and the coccyx region. The so-positioned one or more pocket devices 52 provide a convenient locator mechanism for the operable attachment of one or more force dispersal elements 14 to garment 10 via insertion of such force dispersal elements 14 into respective ones of such pocket devices 52.

Preferably, one or more pocket devices 52 may be selectively disposed on main structure 22 of garment 10 via attachment means (not shown) . Such attachment means may include, for example, adhesives including pressure sensitive adhesive materials disposed on a polymeric backing such as tape strips, hook and loop type fasteners, and the like. Such attachment means may be positioned at one or more major outer surfaces of the respective pocket device 52, such that the user may selectively secure such one or more pocket devices 52 to a respective surface of garment 10. In some embodiments, the attachment means provided in combination with pocket device 52 enables removable engagement of such pocket device 52 to respective surfaces of garment 10. As noted above, it is particularly contemplated by the present invention to secure one or more pocket devices 52 to garment 10 at locations thereof corresponding to injury-prone regions of the user such as the greater trochanter and the coccyx region when garment 10 is properly worn by the user.

In other embodiments of the present invention, one or more pocket devices 52 may be integrally formed with main structure 22 of garment 10 so as to provide a permanent location for the operable reception of one or more force dispersal elements 14 therein. Such integrally formed pocket devices 52 may be configured to removably secure one or more force dispersal elements 14 at least partially therewithin. In such embodiments, pocket device 52 may simply comprise a single sided member that is attached to garment 10 in such a manner so as to define an open cavity between the single sided member and a respective surface of garment 10.

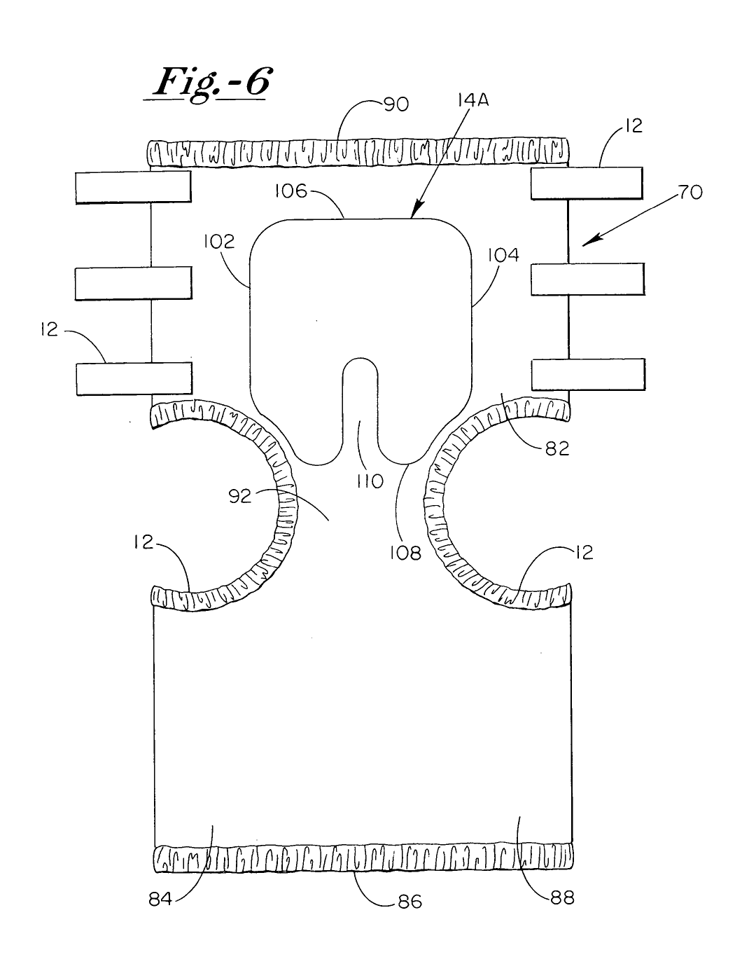

In a further embodiment of the present invention, a wearable garment 70 is provided and is specifically configured for protecting particular user body parts from trauma as a result of external forces focused thereupon. As illustrated in FIG. 6, garment 70 preferably includes a main structure 82 having an inner surface 86, an outer surface 84, and front and rear portions 88, 90 extending from a midsection 92 thereof. A variety of suspension means 12 may be incorporated with garment 70 as described hereinabove.

Garment 70 preferably includes one or more force dispersal elements 14A disposed at rear portion 90 thereof, such that one or more force dispersal elements 14 are operably disposed adjacent the user's coccyx region when garment 70 is properly worn by the user. Though force dispersal elements 14A may be utilized in a variety of configurations, particularly preferred examples of such force dispersal element configurations are described herein with reference to FIGS. 6-9. In a first embodiment of force dispersal element 14A for operable disposition substantially adjacent to the user's coccyx region, and as illustrated in FIG. 6, force dispersal element 14A may be embodied in a single discrete package having first and second substantially parallel longitudinal edges 102, 104, a first transverse edge 106 extending between first and second longitudinal edges 102, 104 along a line substantially perpendicular to first and second longitudinal edges 102, 104. Force dispersal element 14A further includes a second substantially U-shaped transverse edge 108 extending between first and second longitudinal edges 102, 104 and forming an open annulus 110 that is operably aligned in superimposed relationship over the user's coccyx region when garment 70 is properly worn. Force dispersal element 14A is preferably at least about 20 in.² in size, and is more preferably between about 20 in.² and 60 in.² in size.

In a further embodiment, force dispersal element 14B is embodied in two discrete packages each having a substantially elliptical shape with respective major axes 122 and respective minor axes 124. Major axis 122 of force dispersal elements 14B preferably extends substantially along respective longitudinal axes thereof. Force dispersal elements 14B are preferably spaced apart such that a gap 126 defined therebetween is operably aligned in superimposed relationship over a central portion of the user's coccyx region when garment 70 is properly worn. Force dispersal elements 14B are each preferably at least about 6 in.² in size, and more preferably each between about 6 in.² and 30 inc.² in size.

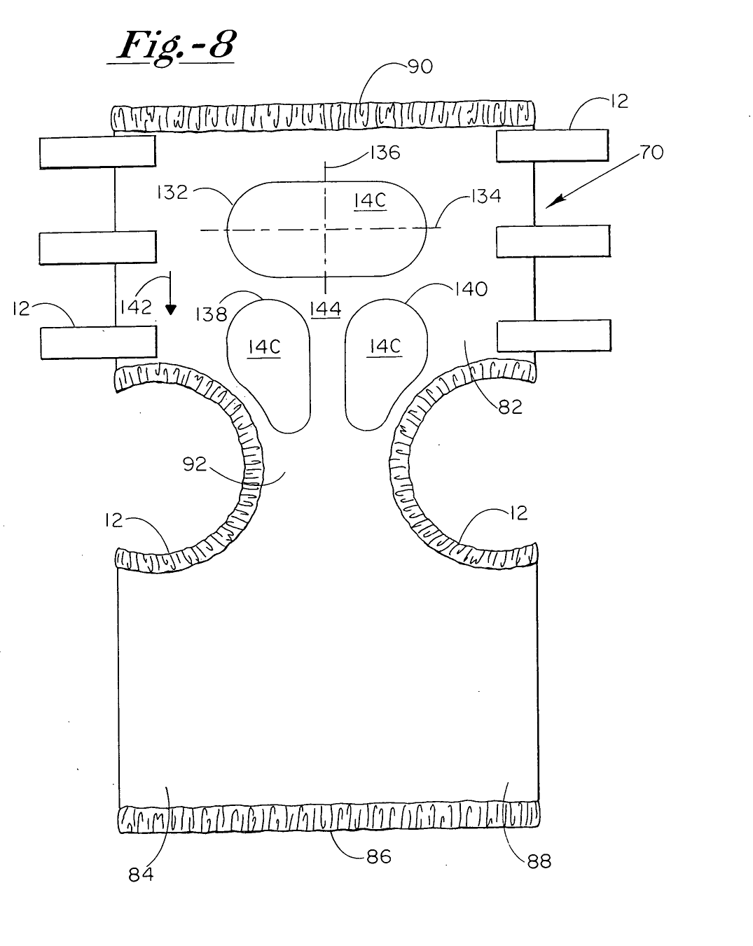

As illustrated in FIG. 8, another embodiment for garment 70 provides for force dispersal element 14C being embodied in three discrete packages. First package 132 of force dispersal element 14C is substantially elliptical in shape with a major axis 134 and a minor axis 136. As shown in FIG. 8, major axis 134 extends substantially along a transverse axis of garment 70. Second and third discrete packages 138, 140 of force dispersal element 14C are preferably substantially circular, or somewhat elliptical, in shape and are transversely spaced from one another and longitudinally spaced from first elliptical package 132 in a first longitudinal direction 142 at garment 70. A gap 144 bound by each of first, second and third discrete packages 132, 138, 140 is operably aligned in superimposed relationship over the user's coccyx region when garment 70 is properly worn. First package 132 is preferably at least 6 in.² in size, and more preferably between about 6 in.² and 30 in.² in size. Second and third packages 138, 140 are each at least about 3 in.² in size, and more preferably between about 3 in.²and about 20 in.² in size.

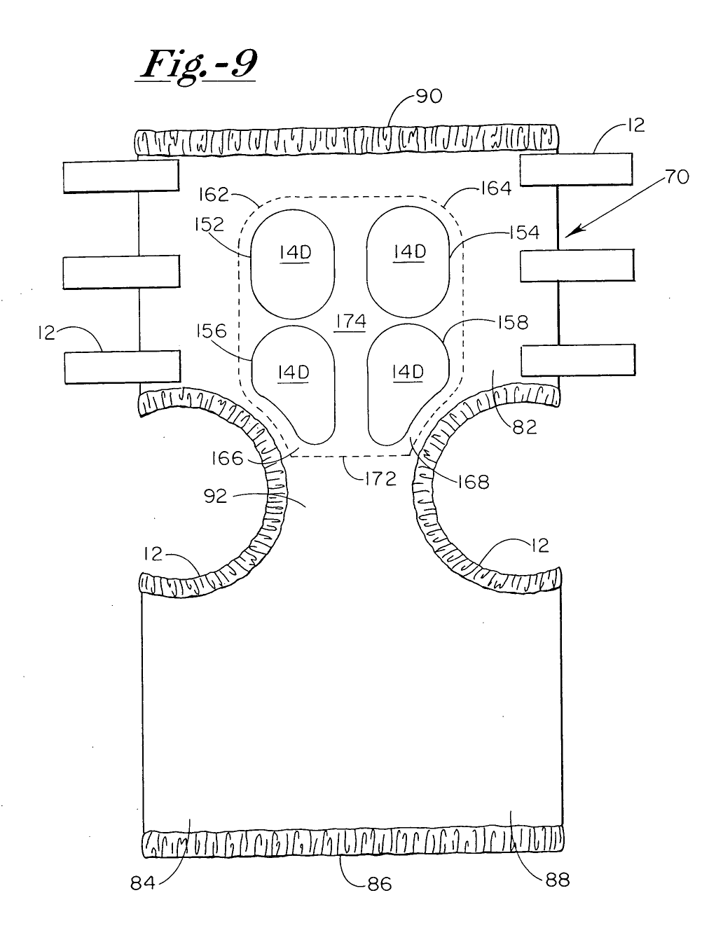

A further embodiment of garment 70 is illustrated in FIG. 9 and includes force dispersal element 14D embodied in four discrete packages 152, 154, 156, and 158. Force dispersal elements 14D are preferably substantially circular, or somewhat elliptical, in shape and are positioned at respective internal corners 162, 164, 166, 168 of an imaginary dimension 172 on garment 70. A gap 174 bound by each of the four discrete packages 152, 154, 156, and 158 is operably aligned in superimposed relationship over a central portion of the user's coccyx region when garment 70 is properly worn. Each of packages 152, 154, 156, and 158 are preferably at least about 3 in.² in size, and more preferably between about 3 in.² and about 20 in.² in size.

As described hereinabove, force dispersal elements 14A-14D may be selectively and/or removably placed at respective positions on garment 70, including inner surface 86 and/or outer surface 84. As such, force dispersal elements 14A-14D may preferably incorporate attachment means (not shown) disposed on one or more major surfaces thereof for providing an operable attachment of such force dispersal elements 14A-14D to respective surfaces of garments 70.

In addition, force dispersal elements 14A-14D may be operably utilized in combination with one or more pocket devices 52 that are specifically configured to receive one or more of such force dispersal elements 14A-14D at least partially therein.

The specific configurations described with respect to force dispersal elements 14A-14D are provided for varying degrees of support and/or flexibility, with certain portions of such force dispersal elements 14A-14D being positioned to absorb and disperse forces directed at respective upper or lower sections of the user's coccyx region.

The embodiments for force dispersal elements 14A-14D illustrated in FIGS. 6-9 for placement at rear portion 90 of garment 70 represent particularly preferred examples of force dispersal element configurations that provide both functionality and comfort to the user. Force dispersal element 14A is configured as a single unit that is positioned and designed to disperse external forces directed at the user's coccyx region. To most effectively do so, force dispersal element 14A provides a cushioning zone toward the rear of force dispersal elements 14A that is effective in dispersing external forces placed upon it when, for example, the user is lying down in a prone position. In addition, force dispersal elements 14A includes a cushioning zone more forwardly placed on garment 70 than the rearwardly placed cushioning zone so as to provide force dispersion for external forces directed at the user's coccyx region when the user is, for example, in a sitting position. Accordingly, force dispersal elements 14A is specifically configured to provide a force dispersal characteristic at least when the user is in a prone position, a sitting position, or positions therebetween.

Additionally, force dispersal elements 14A-14D are preferably configured and disposed at garment 70 to provide an area of garment 70 that is operably aligned with a central portion of the user's coccyx region when garment 70 is properly worn, and which area of garment 70 does not include a portion of force dispersal element 14A disposed thereat. In other words, force dispersal element 14A provides an open gap that is at least partially bound by force dispersal element 14A, which open gap is preferably operably aligned with a central portion of the user's coccyx region. The central portion of the user's coccyx region is generally at and immediately adjacent to the tailbone. The open gap is provided over such a central portion of the user's coccyx region in order to inhibit the imposition of external forces thereat. In other words, forces directed at the user's coccyx region are initially absorbed and redirected about force dispersal element 14A before such forces reach the central portion of the user's coccyx region, in that the thickness of the force dispersal element 14A provides an initial spacing between the central portion of the user's coccyx region and the surface that is applying the external forces to garment 70, and ultimately to the user's coccyx region. In such a manner, the forces applied by such a surface are at least initially absorbed by pertinent regions of force dispersal element 14A and not the central portion of the user's coccyx region. Therefore, the external forces directed at the user's coccyx region are either completely absorbed before the resilient thickness of force dispersal element 14A is depressed to a point at which the central portion of the user's coccyx region comes into contact with the force bearing surface, or such forces have been substantially diminished so as to prevent injury to the central portion of the user's coccyx region when such central portion does come into contact with the force bearing surface.

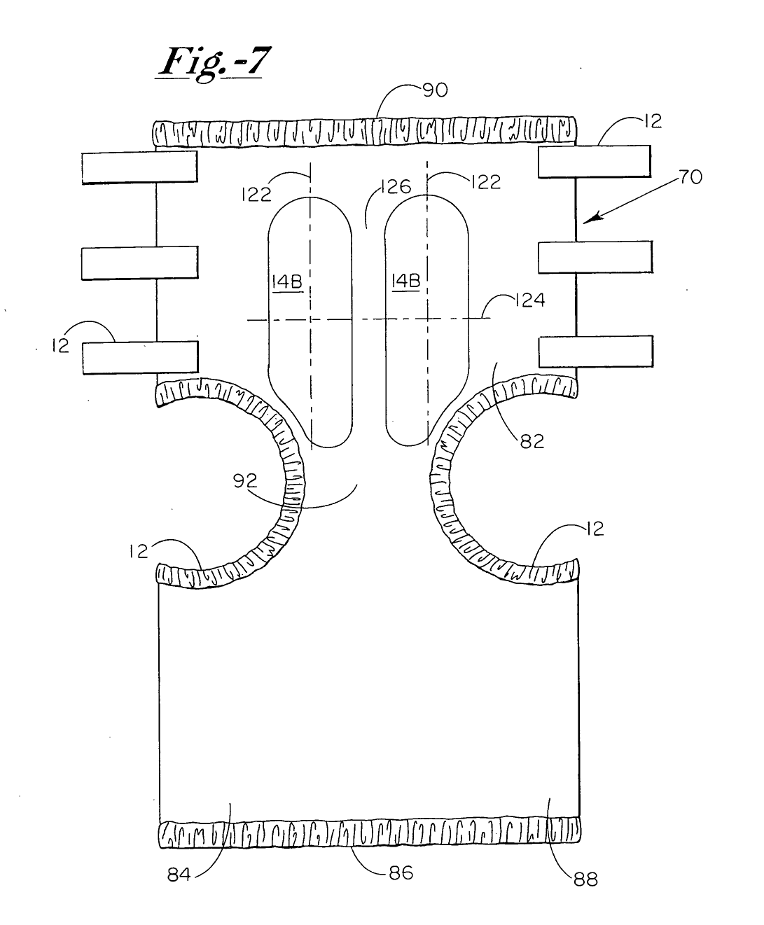

Each of the force dispersal element embodiments 14A-14D described with reference to FIGS. 6-9 incorporate such an open area for preventing and/or inhibiting direct forces placed upon the central portion of the user's coccyx region. For force dispersal element 14A, such an open area is defined by open annulus 110, while the respective open areas defined by force-dispersal element 14B-14D for operable alignment with the central portion of the user's coccyx region are identified in FIGS. 7-9 as open gaps 126, 144, and 174. Further, each of the alternative embodiments illustrated for force dispersal elements 14A-14D provide various degrees of flexibility to garment 70 while still providing support and force dispersal characteristics for the user when the user is in a prone position, a sitting position, and positions therebetween.

A particular application for garment 70 that is enabled and addressed through the configurations of force dispersal elements 14A-14D is in the treatment and prevention of decubitus ulcers. Where users of garment 70 are confined to or choose to be in prone or sitting positions for prolonged periods of time, force dispersal elements 14A-14D are useful for reducing or eliminating continuous pressure placed upon the central portion of the user's coccyx region that can result in decubitus ulcers. As described above, force dispersal elements 14A-14D are specifically configured to reduce or eliminate contact pressure between a force bearing surface and the central portion of the user's coccyx region. In such a manner, the force generated by the weight of the user when in a sitting or lying position is distributed across a relatively large surface area of force dispersal elements 14A-14D, rather than being focused at a central portion of the user's coccyx region.

As mentioned above, the thickness of force dispersal elements 14A-14D plays a role in the effectiveness of reducing or eliminating forces placed upon the central portion of the user's coccyx region. Accordingly, force dispersal elements 14 and force dispersal elements 14A-14D are preferably at least about 0.125 inches thick, and more preferably between about 0.125 and 2.0 inches in thickness. The thickness of such force dispersal elements 14 and 14A-14D depends greatly upon the materials utilized therefor. As such, the present invention contemplates utilizing force dispersal elements 14 and 14A-14D of various thicknesses, so long as force dispersal elements 14 and 14A-14D both provide adequate force dispersal while not being too thick to be cumbersome or uncomfortable to wear.

Although particular sizing dimensions for force dispersal elements 14A-14D are specifically recited above, it is contemplated by the present invention that such dimensions may be altered in order to accommodate garments of extraordinary size. For example, garments intended for use by small individuals such as children or infants, as well as garments intended for use by large adults may require respective force dispersal elements 14 and 14A-14D to be either smaller than, or larger than the dimensions recited above. Accordingly, such dimensions are provided as examples only of the sizes most commonly utilized.

Garment 70 is preferably configured as a wearable garment such as an undergarment that is comfortably wearable by the user under ordinary clothes. Garment 70, however, may be constructed as any desired wearable item that provides protection to injury-prone regions of the user. Accordingly, force dispersal elements 14A-14D may be disposed at any desired surface of garment 70 including inner surface 86 or outer surface 84. In addition, such force dispersal elements 14A-14D may be removably disposed at garment 70, as desired per application.

The invention has been described herein in considerable detail in order to comply with the patent statutes, and to provide those skilled in the art with the information needed to apply the novel principles and to construct and use embodiments of the invention as required. However, it is to be understood that the invention can be carried out by specifically different devices and that various modifications can be accomplished without departing from the scope of the invention itself.

1. An adult diaper for use in operably absorbing excretory materials from the user, said adult diaper further being configured for providing force dispersal to particular body regions of a user, said adult diaper comprising:
(a) a main structure having an inner surface and an outer surface and front and rear portions extending from a mid-section thereof, said main structure being configured to be worn about the user's hips and between the user's legs via suspension means operably coupled to said adult diaper, at least a portion of said main structure being fabricated from one or more layers of fluid-absorbent material; and
(b) force dispersant means being selectively disposed only at one or more predetermined protection locations of said main structure for cushioning external forces focused at one or more of such particular user body regions correspondingly aligned with said one or more protection locations of said main structure when said adult diaper is properly worn by the user.

2. An adult diaper as in claim 1 wherein said force dispersant means is selected from the group consisting of foam pads, rubber pads, selectively inflatable air bladders, closed-cell air pocket sheets, shields, gel pads, and combinations thereof.

3. An adult diaper as in claim 1 wherein the particular user body regions corresponding to said protection locations of said main structure include greater trochanter and coccyx region.

4. An adult diaper as in claim 3 wherein said force dispersant means is disposed at said rear portion of said main structure.

5. An adult diaper as in claim 3 wherein said force dispersant means is disposed at said front portion and said rear portion of said main structure.

6. An adult diaper as in claim 1, including one or more pocket means sized and configured to operably receive respective force dispersant means at least partially therein, said one or more pocket means being disposed at or adjacent to respective ones of said one or more protection locations of said main structure.

7. An adult diaper as in claim 6 wherein said one or more pocket means are adapted to removably receive respective force dispersant means at least partially therein.

8. An adult diaper as in claim 6 wherein said one or more pocket means are removably securable to said main structure.

9. An adult diaper as in claim 6 wherein said one or more pocket means are integrally formed with said main structure of said adult diaper.

10. An adult diaper as in claim 6 wherein said one or more pocket means are fabricated from a liquid-impervious material.

11. An adult diaper as in claim 1 wherein said force dispersant means is a closed-cell air pocket sheet that is selectively secured to said main structure via pressure sensitive adhesive means disposed on at least one major surface of said closed-cell air pocket sheet.

12. An adult diaper as in claim 11, including pressure sensitive adhesive means disposed on both first and second major surfaces of said closed-cell air pocket sheet, wherein said pressure sensitive adhesive means disposed on said first major surface serves to secure a shield to said first surface of said closed-cell air pocket sheet, and the pressure sensitive adhesive means disposed on said second major surface of said closed-cell air pocket sheet serves to selectively secure said closed-cell air pocket sheet to said main structure.

13. A wearable garment that is specifically configured for protecting certain user body parts from trauma as a result of external forces focused thereupon, said garment comprising force dispersant means disposed at a rear portion of the garment adjacent the user's coccyx region when said garment is properly worn, said force dispersant means being embodied in one or more discrete packages distinctly and selectively positioned on a respective surface of said garment and being selected from the group consisting of foam pads, rubber pads, selectively inflatable air bladders, closed-cell air pocket sheets, gel pads, shields, and combinations thereof.

14. A wearable garment as in claim 13 wherein said force dispersant means is disposed only at said rear portion of said garment.

15. A wearable garment as in claim 13 wherein said force dispersant means is embodied in a single discrete package having first and second substantially parallel longitudinal edges, a first transverse edge extending between said first and second longitudinal edges along a line substantially perpendicular to said first and second longitudinal edges, and a second substantially U-shaped transverse edge extending between said first and second longitudinal edges and forming an open annulus that is operably aligned in superimposed relationship over a central portion of the user's coccyx region.

16. A wearable garment as in claim 13 wherein said force dispersant means is embodied in two discrete packages each having a substantially elliptical shape with a major axis and a minor axis, the major axis of each of said elliptical packages extending substantially along a respective longitudinal axis of said garment, said elliptical packages being spaced apart such that a gap defined therebetween is operably aligned in superimposed relationship over a central portion of the user's coccyx region.

17. A wearable garment as in claim 13 wherein said force dispersant means is embodied in three discrete packages, a first of said discrete packages having an elliptical shape with the major axis and a minor axis, the major axis of extending substantially along a transverse axis of said garment, second and third discrete packages being substantially circular and being transversely spaced from one another and longitudinally spaced from said first elliptical package in a first longitudinal direction, such that a gap bound by each of said first, second, and third discrete packages is operably aligned in superimposed relationship over a central portion of the user's coccyx region.

18. A wearable garment as in claim 13 wherein said force dispersant means is embodied in four discrete packages, each of which is substantially circular in shape, each being positioned at a respective internal corner of an imaginary dimension on said garment, such that a gap bound by each of said four discrete packages is operably aligned in superimposed relationship over a central portion of the user's coccyx region.

19. A wearable garment as in claim 13 that is configured as an undergarment.

20. A wearable garment as in any of claims 13 through 18, including one or more pocket means sized and configured to operably receive respective packages of said force dispersant means at least partially therein, said one or more pocket means being disposed at or adjacent to the user's coccyx region.

21. A wearable garment as in claim 20 wherein said one or more pocket means are adapted to removably receive respective packages of said force dispersant means at least partially therein.

22. A wearable garment as in claim 20 wherein said one or more pocket means are removably securable to said garment.

23. A wearable garment as in claim 20 wherein said one or more pocket means are integrally formed with said garment.

24. A wearable garment as in claim 20 wherein said one or more pocket means are fabricated from a liquid-impervious material.
